(19) United States
(12) Patent Application Publication
(10) Pub. No.: US 2019/0262490 A1
(43) Pub. Date: Aug. 29, 2019

(54) COMPOSITE PARTICLES, DISPERSION LIQUID, FILM, DEODORIZING MATERIAL, WET WIPER, AND SPRAY

(71) Applicant: FUJIFILM Corporation, Tokyo (JP)

(72) Inventors: Mitsumasa HAMANO, Minami-ashigara-shi (JP); Tadashi ITO, Minami-ashigara-shi (JP); Naohiro MATSUNAGA, Minami-ashigara-shi (JP); Shigeaki OHTANI, Minami-ashigara-shi (JP)

(73) Assignee: FUJIFILM Corporation, Tokyo (JP)

(21) Appl. No.: 16/411,258

(22) Filed: May 14, 2019

Related U.S. Application Data
(63) Continuation of application No. PCT/JP2017/041096, filed on Nov. 15, 2017.

(30) Foreign Application Priority Data
Nov. 16, 2016 (JP) 2016-223117

(51) Int. Cl. A61L 9/012

(52) U.S. Cl. CPC A61L 9/012

(57) ABSTRACT

An object of the invention is to provide particles that have excellent deodorizing properties but do not easily settle down in a case in which the particles are applied to a dispersion liquid. Another object of the invention is to provide a dispersion liquid and a film, both using the particles; a deodorizing material including the particles, the dispersion liquid, or the film; and a wet wiper and a spray, both including the dispersion liquid.

A composite particle of the invention includes a polymer particle; and at least one kind of inorganic particle selected from the group consisting of metals and metal oxides, the inorganic particle being supported on the surface of the polymer particle, in which the inorganic particles have an average particle diameter of less than 100 nm.

### CROSS-REFERENCE TO RELATED APPLICATIONS

This application is a Continuation of PCT International Application No. PCT/JP2017/041096 filed on Nov. 15, 2017, which claims priority under 35 U.S.C. § 119(a) to Japanese Patent Application No. 2016-223117 filed on Nov. 16, 2016. The above application is hereby expressly incorporated by reference, in its entirety, into the present application.

### BACKGROUND OF THE INVENTION

### 1. Field of the Invention

The present invention relates to composite particles, a dispersion liquid, a film, a deodorizing material, a wet wiper, and a spray.

### 2. Description of the Related Art

In the related art, metal particles and metal oxide particles have been widely used in numerous fields such as the field of electronic printing, the field of powder metallurgy, the field of cosmetics, the field of paint, and the field of resin processing. Particularly, these days, since metal particles and metal oxide particles having particle diameters of several tens of nanometers (nm) or less have relatively strong surface active action, and large specific surface areas, utilization of the particles has also been suggested in the fields of catalyst, adsorbent, and the like.

For example, JP2015-190071A discloses “an aqueous dispersion characterized in that metal chelate microparticles having an average particle diameter of 50 nm or less are dispersed in water” as an aqueous dispersion of metal chelate microparticles having antibacterial and deodorant performance.

### SUMMARY OF THE INVENTION

The inventors of the present invention produced a dispersion liquid in which inorganic particles selected from the group consisting of metal particles and metal oxide particles are dispersed in a solvent and conducted an investigation on the deodorizing properties of the dispersion liquid, and the inventors found that there are occasions in which the inorganic particles are likely to settle down in the dispersion liquid. In a case in which inorganic particles settle down in a dispersion liquid, the inorganic particles thus settled aggregate, and the specific surface area thereof becomes small. As the result, deterioration of the deodorizing properties of the dispersion liquid may be brought. Furthermore, it was discovered that even among inorganic particles, particles having an average particle diameter of less than 100 nm have excellent deodorizing properties due to the size effect of the particles; however, these particles have a noticeable tendency to cause excessive growth or aggregation, and it is difficult to obtain a stable deodorizing effect.

Therefore, there is a demand to provide particles that have excellent deodorizing properties but do not easily settle down in a case in which the particles are applied to a dispersion liquid (in other words, having excellent resistance to settling).

Thus, an object of the invention is to provide particles that have excellent deodorizing properties but do not easily settle down in a case in which the particles are applied to a dispersion liquid.

Furthermore, another object of the invention is to provide a dispersion liquid and a film, both of which use the particles.

Still another object of the invention is to provide a deodorizing material including the particles, the dispersion liquid, or the film.

Still another object of the invention is to provide a wet wiper and a spray, both of which include the dispersion liquid.

The inventors conducted a thorough investigation in order to achieve the problems described above, and as a result, the inventors found that the objects can be achieved by supporting predetermined inorganic particles having an average particle diameter of less than 100 nm on polymer particles, thus completing the invention.

That is, the inventors found that the objects described above can be achieved by the following configurations.

(1) A composite particle comprising:

a polymer particle; and

at least one kind of inorganic particle selected from the group consisting of metal particles and metal oxide particles, the inorganic particle being supported on the surface of the polymer particle,

wherein the inorganic particles have an average particle diameter of less than 100 nm.

(2) The composite particle according to (1), further comprising a coating film formed from a silane compound on at least a portion of the surface of the polymer particle.

(3) The composite particle according to (1) or (2), wherein the inorganic particle includes at least one selected from the group consisting of Cu, Ag, Zn, Ti, Ni, W, Sn, Fe, Sr, Bi, and Mn.

(4) The composite particle according to any one of (1) to (3), wherein the polymer particles have an average particle diameter of more than 50 nm.

(5) The composite particle according to any one of (1) to (4), wherein the polymer particles have an average particle diameter of 100 to 800 nm.

(6) The composite particle according to any one of (1) to (5), wherein the polymer particle includes, as a resin material that constitutes the polymer particle, at least one selected from the group consisting of an acrylic resin, a methacrylic resin, a polystyrene resin, a polyolefin resin, and a copolymer formed from a polystyrene resin and an acrylic resin or a methacrylic resin.

(7) A dispersion liquid comprising:

the composite particle according to any one of (1) to (6); and

a solvent.

(8) The dispersion liquid according to (7), further comprising a thermoplastic resin or a silicate-based compound.

(9) A film comprising:

the composite particle according to any one of (1) to (6); and

a binder. (10) A deodorizing material comprising the composite particle according to any one of (1) to (6), the dispersion liquid according to (7) or (8), or the film according to (9).

(11) A wet wiper comprising:

a base fabric; and

the dispersion liquid according to (7) or (8) impregnated into the base fabric.

(12) A spray comprising:

a spray container; and

the dispersion liquid according to (7) or (8) stored in the spray container.

According to the invention, particles that have excellent deodorizing properties but do not easily settle down in a case in which the particles are applied to a dispersion liquid, can be provided.

Furthermore, according to the invention, a dispersion liquid and a film, both of which use the particles, can be provided.

According to the invention, a deodorizing material including the particles, the dispersion liquid, or the film can be provided.

According to the invention, a wet wiper and a spray, both of which include the dispersion liquid, can be provided.

### BRIEF DESCRIPTION OF THE DRAWINGS

FIG. 1 is an optical microscopic photograph of composite particles 1.

### DESCRIPTION OF THE PREFERRED EMBODIMENTS

The composite particles, the dispersion liquid, the film, the deodorizing material, the wet wiper, and the spray according to the embodiment of the invention will be described in detail below.

The explanation of the constitution requirements that will be described below is based on representative embodiments of the invention; however, the invention is not intended to be limited to such embodiments.

Meanwhile, with regard to a substituent for which substitution or unsubstitution is not specified in the present specification, the group may be further substituted with a substituent to the extent that does not impair the intended effect. For example, the description of “alkyl group” corresponds to an alkyl group which may be substituted with a substituent.

In the present specification, a numerical range represented using the symbol “˜” means a range including the numerical values described before and after the symbol “˜” as the lower limit and the upper limit.

Furthermore, in the present specification, the term “(meth)acryl” represents both or any one of acryl or methacryl.

[Composite Particle]

A composite particle according to the embodiment of the invention comprise a polymer particle; and at least one kind of inorganic particle selected from the group consisting of metal particles and metal oxide particles, the inorganic particle being supported on the surface of the polymer particle and having an average particle diameter of less than 100 nm.

Polymer particles generally have a feature of having excellent dispersibility compared to inorganic particles. In the composite particles according to the embodiment of the invention, since inorganic particles having an average particle diameter of less than 100 nm, which are likely to cause excessive growth or aggregation as described above, are supported on polymer particles, the composite particles do not easily settle down even in a dispersion liquid. Furthermore, due to the structural feature, the inorganic particles are inhibited from undergoing excessive growth or aggregation in the dispersion liquid and are maintained in a state of having a large specific surface area and an average particle diameter of less than 100 nm. Therefore, the composite particles exhibit stable deodorizing properties.

The term “support” refers to a state in which polymer particles are used as a carrier, and metal particles and/or metal oxide particles are fixed thereon. At this time, the metal particles and/or metal oxide particles may or may not be in direct contact with the surface of the polymer particles, as will be described below.

Regarding the composite particles, the structure is not particularly limited as long as the inorganic particles are supported on the surface of the polymer particles. Above all, from the viewpoint of obtaining superior effects of the invention, it is preferable that the composite particles have a coating film formed from a silane compound that will be described below on at least a portion (one region) of the surface of the polymer particle.

Examples of the aspect of having a coating film formed from a silane compound on at least a portion of the surface of the polymer particle include an aspect in which a coating film of a silane compound is disposed so as to cover the surface of polymer particles having inorganic particles supported in a contacting state such that each of the inorganic particles is covered, and an aspect in which a coating film of a silane compound is disposed on the surface of polymer particles having inorganic particles supported in a contacting state such that the inorganic particles are not completely covered (in other words, the inorganic particles are not embedded in the coating film of the silane compound). An aspect in which a coating film of a silane compound is disposed on the surface of polymer particles, and inorganic particles are supported on the surface of the polymer particles through the coating film of the silane compound is also acceptable.

The coating film of a silane compound may be disposed over the entire region on the surface of the polymer particle or may be disposed in one region.

Hereinafter, various components that constitute the composite particles according to the embodiment of the invention will be described in detail.

<Inorganic Particles>

The composite particles have at least one kind of inorganic particle selected from the group consisting of metal particles and metal oxide particles, which has an average particle diameter of less than 100 nm.

The type of the inorganic particles is not particularly limited, and any known metal particles and metal oxide particles having a deodorizing effect can be used. For example, particles that exhibit a deodorizing effect for hydrogen sulfide, amine-like odor, ammonia, age-related odor (nonenal, isovaleric acid, and the like), acetic acid, methylmercaptan, and the like can be suitably used.

Among them, it is preferable that the inorganic particles include one selected from the group consisting of Cu, Ag, Zn, Ti, Ni, W, Sn, Fe, Sr, Bi, and Mn, and from the viewpoint of having superior deodorizing properties, it is more preferable that the inorganic particles include one selected from the group consisting of Cu, Ag, and Zn.

More specifically, it is preferable that the inorganic particles are particles of a metal selected from the group consisting of Cu, Ag, Zn, Ti, Ni, W, Sn, Fe, Sr, Bi, and Mn, or oxide particles of a metal selected from the group consisting of Cu, Ag, Zn, Ti, Ni, W, Sn, Fe, Sr, Bi, and Mn; more preferably particles of a metal selected from the group consisting of Cu, Ag, and Zn, or oxide particles of a metal selected from the group consisting of Cu, Ag, and Zn; and even more preferably oxide particles of a metal selected from the group consisting of Cu and Zn.

In the composite particles, one kind of inorganic particle may be used alone, or two or more kinds of inorganic particles may be used in combination.

The average particle diameter of the inorganic particles is less than 100 nm.

The average particle diameter of the inorganic particles can be measured by observing the composite particles using an electron microscope. Specifically, the average particle diameter is a value obtained by measuring the diameters of primary particles and secondary particles (here, the “secondary particles” are defined as aggregates composed of primary particles that are fused or come into contact) for the inorganic particles in the composite particles from electron microscopic images, and averaging the diameters of particles in the range of 90%, with 5% of the number of particles on the side of smallest diameters and 5% of the number of particles on the side of largest diameters being excluded from the total number of particles. Here, the diameter refers to a diameter corresponding to the circumscribed circle diameter of a particle.

In a case in which there is no significant change in the particle shape between the inorganic particles in the composite particles from an electron microscopic image and the inorganic particles in a state in which only the inorganic particles are dispersed, measured values obtained by dynamic light scattering using a dispersion liquid of the inorganic particles only can be used in substitution for the average particle diameter. In this case, the average particle diameter can be measured by dynamic light scattering using a particle size distribution analyzer based on laser diffraction, or the like.

In the present specification, the “average particle diameter” was measured using a dynamic light scattering measuring apparatus (ZETASIZER ZS) manufactured by Malvern Panalytical Ltd. The average particle diameter was measured three times as the average value (Z-Average) of the particle diameter based on a cumulant analysis by the technique defined in ISO13321, and the average value of the values measured three times was used.

For the reason of obtaining superior deodorizing properties, the average particle diameter of the inorganic particles is preferably 90 nm or less, more preferably 70 nm or less, and even more preferably 50 nm or less. The lower limit is not particularly limited; however, for example, the lower limit is 1 nm or more.

The average primary particle diameter of the inorganic particles is preferably less than 100 nm. The lower limit is not particularly limited; however, for example, the lower limit is 1 nm or more. For the reason of obtaining superior deodorizing properties, the average primary particle diameter of the inorganic particles is more preferably 5 to 90 nm, and even more preferably 5 to 50 nm.

The “average primary particle diameter” is a value obtained by measuring the diameters of various primary particles from electron microscopic images, and averaging the diameters of primary particles in the range of 90%, with 5% of the number of primary particles on the side of smallest diameters and 5% of the number of primary particles on the side of largest diameters being excluded from the total number of primary particles. Here, the diameter refers to a diameter corresponding to the circumscribed circle diameter of a primary particle.

The shape of the inorganic particles (metal particles and metal oxide particles) is not particularly limited as long as the shape is a particulate shape, and examples thereof include a spherical shape, an elliptical shape, a rod shape, and a plate shape. It is not necessary for the inorganic particles to be perfect spheres, ellipsoids, or the like, and some particles may be distorted. It is more advantageous for the inorganic particles to be spherical, rather than a rod shape or a plate shape, because the contact area between the inorganic particles is reduced, and it becomes difficult for the inorganic particles to aggregate.

The average particle diameter of the inorganic particles can be regulated by a method known in the related art, and for example, dry pulverization or wet pulverization can be employed. For dry pulverization, for example, a mortar, a jet mill, a hammer mill, a pin mill, a tumbling mill, a vibrating mill, a planetary mill, and a beads mill are used as appropriate. Furthermore, for wet pulverization, for example, various ball mills, a high-speed rotary crusher, a jet mill, a beads mill, an ultrasound homogenizer, a high-pressure homogenizer, and the like are used as appropriate.

For example, in a beads mill, the average particle diameter can be controlled by regulating the diameter, type, and the mixing amount of the beads that serve as media, and the like.

In the invention, for example, the average particle diameter of the inorganic particles can be regulated by wet pulverization by dispersing inorganic particles (metal particles and metal oxide particles) to be pulverized in ethanol or water, mixing the dispersion with zirconia beads having different sizes, and vibrating the mixture. However, the method is not limited to this method, and any method appropriate for controlling the particle diameter may be selected.

In order to obtain a desired particle size distribution, wet-pulverized particles may be screened. Examples of screening include a screening method of utilizing the difference in the settling speed of particles (water sieving), and a method of using a membrane filter.

<Polymer Particles>

The composite particles include polymer particles as a carrier.

The type of the polymer particles is not particularly limited, and any known polymer particles can be used.

Examples of the resin material that constitutes the polymer particles include a polyurethane resin, a polyester resin, a (meth)acrylic resin, a polystyrene resin, a polyolefin resin, a fluororesin, a melamine resin, a vinyl resin, a polystyrene-(meth)acrylic copolymer resin, a polyimide resin, a fluorinated polyimide resin, a polyamide resin, a polyamideimide resin, a polyetherimide resin, a cellulose acylate resin, a polyether ether ketone resin, a polycarbonate resin, an alicyclic polyolefin resin, a polyallylate resin, a polyethersulfone resin, a polysulfone resin, a resin formed from a cycloolefin copolymer, a fluorene ring-modified polycarbonate resin, an alicyclic-modified polycarbonate resin, and a fluorene ring-modified polyester resin.

Above all, it is preferable that the polymer particles include, as a resin material that constitutes the polymer particles, at least one selected from the group consisting of a polyurethane resin, a (meth)acrylic resin, a polystyrene resin, a polystyrene-(meth)acrylic copolymer resin, and a polyolefin resin, and it is more preferable that the polymer particles include at least one selected from the group consisting of a (meth)acrylic resin, a polystyrene resin, a polyolefin resin, and a polystyrene-(meth)acrylic copolymer resin. Meanwhile, a polystyrene-(meth)acrylic copolymer resin means a copolymer formed from a polystyrene resin and an acrylic resin or a methacrylic resin.

Examples of polymer particles including a (meth)acrylic resin as a resin material include EPOSTAR 050W and 100W manufactured by Nippon Shokubai Co., Ltd.; MP-1000, MP-2800, MX-80H3wT, and MX-150 manufactured by Soken Chemical & Engineering Co., Ltd.

Examples of polymer particles including a polyolefin resin as a resin material include AROBASE SE-1013N manufactured by Unitika, Ltd.

Examples of polymer particles including a polystyrene resin as a resin material include SX8743(C)-03 manufactured by JSR Corporation.

Examples of polymer particles including a polystyrene-(meth)acrylic copolymer resin as a resin material include BONRON PS-002 manufactured by Mitsui Chemicals, Inc.

In the composite particles, one kind of polymer particles may be used alone, or two or more kinds of polymer particles may be used in combination.

The average particle diameter of the polymer particles is not particularly limited; however, from the viewpoint of obtaining superior effects of the invention, the average particle diameter is preferably more than 50 nm, and more preferably 60 nm or more. The upper limit of the average particle diameter of the polymer particles is not particularly limited; however, for example, the upper limit is 5,000 nm or less. From the viewpoint of obtaining superior resistance to settling of the composite particles, the average particle diameter of the polymer particles is preferably 100 to 1,000 nm, and more preferably 100 to 800 nm.

Meanwhile, the average particle diameter of the polymer particles is desirably 100 nm or more, from the viewpoint that an effect of substantially reducing the nanoparticle safety risk against metallic substances can be further expected.

The average particle diameter of the polymer particles can be measured by a method similar to that for the average particle diameter of the inorganic particles as described above.

With regard to the composite particles, the ratio of the polymer particles and the inorganic particles is not particularly limited; however, from the viewpoint of obtaining superior resistance to settling, the ratio is preferably, for example, in the range of 1/0.00001 to 1/100,000, and more preferably in the range of 1/0.0001 to 1/10,000, as a mass ratio.

<Silane Compound>

It is preferable that the composite particles have a coating film formed from a silane compound on at least a portion of the surface of the polymer particle.

A silane compound is a compound containing silicon atoms. Above all, from the viewpoint of obtaining superior effects of the invention, it is preferable that the silane compound is a silicone resin formed from an organosiloxane unit.

Meanwhile, usually, organosiloxane units are classified based on how many silicon atoms a monovalent organic group represented by a methyl group or a phenyl group is bonded to, and the organosiloxane units include a bifunctional organosiloxane unit called unit D, to which two organic groups are bonded; a trifunctional organosiloxane unit called unit T, to which one organic group is bonded; a monofunctional organosiloxane unit called unit M, to which three organic groups are bonded; a tetrafunctional organosiloxane unit called unit Q, which has no organic group; and the like.

The unit Q is a unit that does not have any organic group (an organic group having a carbon atom bonded to a silicon atom) bonded to a silicon atom; however, in the invention, the unit Q is regarded as an organosiloxane unit.

The coating film of a silane compound can be formed using, for example, a compound represented by General Formula (1′).

In the following description, the compound represented by General Formula (P) will be described in detail.

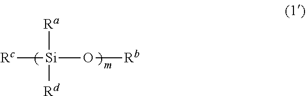

In General Formula (1′), R^{a}, R^{b}, R^{c}, and R^{d} each independently represent a hydrogen atom or an organic group. Furthermore, m represents an integer from 1 to 100. Meanwhile, R^{a} to R^{d} may be respectively bonded to one another and form a ring.

Examples of the organic group represented by R^{a} to R^{d} include an alkyl group having 1 to 20 carbon atoms, an aryl group having 6 to 20 carbon atoms, an alkoxy group having 1 to 20 carbon atoms, and a heterocyclic group having 4 to 16 carbon atoms.

R^{a} to R^{d} are each preferably a hydrogen atom, an alkyl group having 1 to 12 carbon atoms, an aryl group having 6 to 14 carbon atoms, or an alkoxy group having 1 to 12 carbon atoms; and more preferably a hydrogen atom, an alkyl group having 1 to 6 carbon atoms, an aryl group having 6 to 10 carbon atoms, or an alkoxy group having 1 to 6 carbon atoms. The alkyl group represented by R^{a} to R^{d} may be any one of a linear group, a branched group, or a cyclic group. The organic group represented by R^{a} to R^{d} may have a substituent, and this substituent may further have a substituent.

Specific examples of R^{a} to R^{d} include a hydrogen atom, a methyl group, an ethyl group, a propyl group, a butyl group, an isopropyl group, an n-butyl group, a tert-butyl group, an n-pentyl group, an n-hexyl group, a cyclohexyl group, a phenyl group, a naphthyl group, a methoxy group, and an ethoxy group.

m is preferably 2 to 20, more preferably 3 to 15, and even more preferably 5 to 10.

From the viewpoint of obtaining a coating film exhibiting hydrophilicity, the compound represented by General Formula (1′) is preferably such that R^{a}, R^{b}, R^{c}, or R^{d} is a hydrolyzable group (for example, an alkoxy group), and it is more preferable that the compound represented by General Formula (1′) is a silicate-based compound represented by General Formula (1).

In the present specification, the “silicate-based compound” is a compound selected from the group consisting of a compound in which a hydrolyzable group is bonded to a silicon atom, a hydrolysate of the compound, and a hydrolysis condensation product of the compound, and for example, the silicate-based compound may be at least one selected from the group consisting of a compound represented by General Formula (1), a hydrolysate of the compound, and a hydrolysis condensation product of the compound.

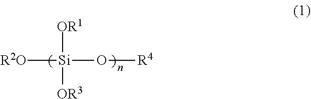

In General Formula (1), R¹, R², R³, and R⁴ each independently represent an organic group having 1 to 6 carbon atoms. Furthermore, n represent an integer from 1 to 100.

The organic group represented by R¹ to R⁴ is preferably an alkyl group having 1 to 6 carbon atoms. Furthermore, the alkyl group having 1 to 6 carbon atoms represented by R¹ to R⁴ may be any one of a linear group, a branched group, or a cyclic group. Examples of the alkyl group having 1 to 6 carbon atoms represented by R¹ to R⁴ include a methyl group, an ethyl group, a propyl group, an isopropyl group, an n-butyl group, a tert-butyl group, an n-pentyl group, an n-hexyl group, and a cyclohexyl group. In a case in which the organic group represented by R¹ to R⁴ is an alkyl group having 1 to 6 carbon atoms, hydrolyzability of the silicate-based compound can be enhanced. In view of the ease of hydrolysis, the alkyl group having 1 to 6 carbon atoms represented by R¹ to R⁴ is more preferably an alkyl group having 1 to 4 carbon atoms, and even more preferably an alkyl group having 1 or 2 carbon atoms.

In General Formula (1), n is preferably an integer from 2 to 100. When n is 100 or less, the viscosity of a solution including a hydrolysate can be adjusted to an appropriate range. Furthermore, when n is 2 or greater, the reactivity of the silicate-based compound can be controlled to be in a preferable range, and satisfactory hydrophilicity is exhibited after application. n is more preferably 3 to 15, and even more preferably 5 to 10.

The compound represented by General Formula (1) is not particularly limited; however, examples thereof include tetramethyl silicate, tetraethyl silicate, tetra-n-propyl silicate, tetra-i-propyl silicate, tetra-n-butyl silicate, tetra-i-butyl silicate, tetra-t-butyl silicate, methyl ethyl silicate, methyl propyl silicate, methyl butyl silicate, ethyl propyl silicate, and propyl butyl silicate.

The compounds represented by General Formula (1) may be used singly or in combination of two or more kinds thereof.

As the compound represented by General Formula (1) is mixed together with a water component, at least a portion is brought to a hydrolyzed state. A hydrolysate of the compound represented by General Formula (1) is obtained by reacting a compound represented by General Formula (1) with a water component, and converting the alkoxy group bonded to silicon to a hydroxy group. On the occasion of hydrolysis, it is not essentially necessary for all of the alkoxy groups to react; however, in order to exhibit hydrophilicity after application, it is preferable that as many alkoxy groups as possible are hydrolyzed. Furthermore, the least amount of the water component needed upon hydrolysis is a molar amount equal to that of alkoxy groups of the compound represented by General Formula (1); however, in order to allow the reaction to proceed smoothly, it is preferable that a large excess amount of water is present.

Here, the “hydrolysate of the compound represented by General Formula (1)” means a compound obtainable as a result of hydrolysis of an OR group (R: R¹ to R⁴) in the compound represented by General Formula (1). Meanwhile, the hydrolysate may be a product obtainable as all of OR groups have been hydrolyzed (complete hydrolysate), or may be a product obtainable as a portion of OR groups have been hydrolyzed (partial hydrolysate). That is, the hydrolysate may be a complete hydrolysate, a partial hydrolysate, or a mixture thereof.

Furthermore, the “hydrolysis condensation product of the compound represented by General Formula (1)” means a compound obtainable by hydrolyzing an OR group (R: R¹ to R⁴) in the compound represented by General Formula (1) and condensing the hydrolysate thus obtained. Meanwhile, the hydrolysis condensation product may be a product obtainable as all of the OR groups have been hydrolyzed, while the hydrolysate has been entirely condensed (complete hydrolysis condensation product), or may be a product obtainable as a portion of OR groups have been hydrolyzed, and a portion of the hydrolysate has been condensed (partial hydrolysis condensation product). That is, the hydrolysis condensation product may be a complete hydrolysis condensation product, a partial hydrolysis condensation product, or a mixture thereof.

A hydrolysis reaction of the compound represented by General Formula (1) proceeds even at room temperature; however, the reaction system may be heated in order to accelerate the reaction. It is preferable that the reaction time is longer because the reaction proceeds further. Furthermore, it is possible to obtain a hydrolysate in about half a day in the presence of a catalyst.

Generally, a hydrolysis reaction is a reversible reaction, and in a case in which water is excluded from the system, a hydrolysate of the compound represented by General Formula (1) initiates condensation between hydroxy groups. Therefore, in a case in which the compound represented by General Formula (1) is caused to react with a large excess of water, and an aqueous solution of a hydrolysate is obtained, it is preferable to use the aqueous solution directly as an aqueous solution without forcibly isolating the hydrolysate therefrom.

As the compound represented by General Formula (1), a commercially available product can be used, and specific examples thereof include “ETHYL SILICATE 48” manufactured by Colcoat Co., Ltd., and MKC (registered trademark) silicate manufactured by Mitsubishi Chemical Corporation.

<Method for Producing Composite Particles>

The method for producing the composite particles is not particularly limited, and for example, a method of using water, an alcohol, and the like as a solvent, adding the above-mentioned polymer particles, the compound represented by General Formula (1), and the inorganic particles at a predetermined amount ratio, and stirring the mixture, may be mentioned. The composite particles may further have other components (for example, additives that will be described below) as necessary to the extent that does not impair the purpose of the invention.

After the production method described above, the composite particles thus obtained may be isolated by centrifugation, or without isolating the composite particles thus obtained, the solution used in the above-described reaction may be used as a dispersion liquid that will be described below.

[Dispersion Liquid]

A dispersion liquid according to the embodiment of the invention includes the composite particles and a solvent.

In the following description, various components that constitute the dispersion liquid according to the embodiment of the invention will be explained, and the physical properties of the dispersion liquid will be described in detail.

<Composite Particles>

As the composite particles, the above-mentioned particles can be used.

It is preferable that the content of the composite particles in the dispersion liquid is adjusted to be 50% by mass or less with respect to the total mass of the dispersion liquid. From the viewpoint of having superior resistance to settling, it is preferable to adjust the content to be 40% by mass or less. The lower limit is not particularly limited; however, for example, the lower limit is 0.000001% by mass or more.

It is preferable that the inorganic particles in the composite particles are adjusted to be 30% by mass or less with respect to the total mass of the dispersion liquid. From the viewpoint of obtaining superior resistance to settling, with respect to the total mass of the dispersion liquid, it is more preferable that the content is adjusted to be 20% by mass or less and it is even more preferable that the content is adjusted to be 15% by mass or less. The lower limit is not particularly limited; however, for example, the lower limit is 0.000001% by mass or more.

In the dispersion liquid, the composite particles may become dispersed particles, which are obtained by aggregating a portion of the composite particles and dispersing the particles. The average particle diameter of the composite particles in the dispersion liquid can be measured by dynamic light scattering using a particle size distribution analyzer based on laser diffraction, or the like. Regarding the method for measuring by dynamic light scattering, specifically, the average particle diameter can be measured by a method similar to that for the average particle diameter of the inorganic particles as described above.

From the viewpoint of having excellent dispersibility and capable of suppressing settling of aggregates, the average particle diameter of the composite particles in the dispersion liquid is preferably 6,000 nm or less, more preferably 50 to 5,000 nm, and even more preferably 100 to 3,000 nm.

<Solvent>

The solvent that is included in the dispersion liquid is not particularly limited, and examples thereof include water, an organic solvent, and a mixture of water and an organic solvent.

Examples of the organic solvent include alcohol-based solvents such as a lower alcohol having 1 to 6 carbon atoms (specifically, examples thereof include methanol, ethanol, n-propanol, i-propanol, n-butanol, 2-butanol, i-butanol, sec-butanol, t-butanol, n-pentanol, t-amyl alcohol, and n-hexanol. Among them, methanol, ethanol, isopropanol, butanol, or n-propanol is preferred, and ethanol or isopropanol is more preferred.), a higher alcohol having 7 or more carbon atoms (preferably, 7 to 15 carbon atoms) (specifically, examples thereof include capryl alcohol, lauryl alcohol, and myristyl alcohol.), phenyl ethyl alcohol, and ethylene glycol; glycol ether-based solvents such as methyl cellosolve, ethyl cellosolve, ethylene glycol dimethyl ether, ethylene glycol diethyl ether, propylene glycol monomethyl ether, propylene glycol monoethyl ether, propylene glycol monopropyl ether, propylene glycol dimethyl ether, ethylene glycol mono-n-butyl ether, diethylene glycol mono-n-butyl ether, triethylene glycol mono-n-butyl ether, tetraethylene glycol mono-n-butyl ether, dipropylene glycol monobutyl ether, and propylene glycol diethyl ether; aromatic hydrocarbon-based solvents such as benzene, toluene, xylene, and ethylbenzene; alicyclic hydrocarbon-based solvents such as cyclopentane, cyclohexane, methylcyclohexane, and ethylcyclohexane; ether-based solvents such as diethyl ether, tetrahydrofuran, dioxane, diisopropyl ether, and di-n-butyl ether; ketone-based solvents such as acetone, methyl ethyl ketone, and methyl isobutyl ketone; and ester-based solvents such as methyl acetate, ethyl acetate, n-propyl acetate, isopropyl acetate, n-butyl acetate, isobutyl acetate, n-amyl acetate, isoamyl acetate, hexyl acetate, ethyl propionate, and butyl propionate.

Furthermore, in addition to the organic solvents described above, examples of the organic solvent include a 10% denatonium benzoate alcohol solution, hexane, geraniol, octaacetylated sucrose, brucine, linalool, linalyl acetate, and acetic acid.

Among the organic solvents, from the viewpoint of killing a wide variety of microorganisms in a short time, particularly an alcohol-based solvent and a glycol ether-based solvent are preferred.

Furthermore, it is preferable to use pure water as the water.

Regarding the solvent, among those described above, water, an alcohol-based solvent, or a glycol ether-based solvent is preferred; water or an alcohol-based solvent is more preferred; and water is even more preferred.

Meanwhile, the solvents may be used singly, or two or more kinds thereof may be used in combination.

In a case in which the solvent includes a hydrophilic organic solvent other than an alcohol-based solvent and a glycol ether-based solvent, the content of the hydrophilic organic solvent other than the alcohol-based solvent and the glycol ether-based solvent is preferably 40% by mass or less with respect to the total mass of the solvent.

<Other Components>

The dispersion liquid can include other additives as necessary, to the extent that does not impair the purpose of the invention.

Examples of the additives include known additives such as an ultraviolet absorber, a preservative, a pH adjusting agent, an anti-foaming agent, a catalyst, a photocatalytic material, a surfactant, a filler, an anti-aging agent, an antistatic agent, a flame retardant, an acidic agent, an alkaline agent, a tackifier, an antioxidant, a leveling agent, a matting agent, a photostabilizer, a dye, a pigment, a dispersant, a fragrance, a film-forming agent, and a dispersion stabilizer. Among them, it is preferable that a dispersant or a film-forming agent is included. In the following description, a dispersant and a film-forming agent will be described in detail.

(Dispersant)

The dispersant as an additive is not particularly limited, and for example, an anionic dispersant, a cationic dispersant, an amphoteric ionic dispersant, and a nonionic dispersant can all be used. Furthermore, the dispersant may be a low-molecular weight compound or a polymer. Regarding the dispersant, above all, sodium hexametaphosphate is preferred.

The content of the dispersant in the dispersion liquid may be regulated as appropriate according to the type of the dispersant or the like; however, for example, the content is preferably 0% to 10% by mass, and more preferably 0% to 8% by mass, with respect to the total solid content of the dispersion liquid.

(Film-Forming Agent)

Examples of the film-forming agent as an additive include a thermoplastic resin and a silicate compound. The film-forming agent functions, for example, in a case in which a film that will be described below is formed, as a binder for causing the composite particles to be fixed to a base material. That is, in a case in which a thermoplastic resin or a silicate compound is used as the film-forming agent, a thermoplastic resin or a compound having a siloxane bond that is formed by the silicate compound functions as the binder.

<<Thermoplastic Resin>>

The thermoplastic resin is preferably a resin having a lowest film-forming temperature of 0° C. to 35° C., and any known thermoplastic resin can be used. Examples thereof include a polyurethane resin, a polyester resin, a (meth)acrylic resin, a polystyrene resin, a fluororesin, a polyimide resin, a fluorinated polyimide resin, a polyamide resin, a polyamideimide resin, a polyetherimide resin, a cellulose acylate resin, a polyurethane resin, a polyether ether ketone resin, a polycarbonate resin, an alicyclic polyolefin resin, a polyallylate resin, a polyethersulfone resin, a polysulfone resin, a resin formed from a cycloolefin copolymer, a fluorene ring-modified polycarbonate resin, an alicyclic modified polycarbonate resin, and a fluorene ring-modified polyester resin. Among them, a (meth)acrylic resin or a urethane resin is preferred.

The thermoplastic resins may be used singly, or two or more kinds thereof may be used in combination.

The content of the thermoplastic resin in the dispersion liquid may be regulated as appropriate according to the type of the thermoplastic resin or the like; however, for example, the content is preferably 0% to 90% by mass, and more preferably 0% to 80% by mass, with respect to the total solid content of the dispersion liquid.

<<Silicate-Based Compound>>

The silicate-based compound is not particularly limited, and for example, a compound represented by General Formula (1) described above may be mentioned.

Regarding the silicate-based compound, one kind may be used alone, or two or more kinds may be used in combination.

The content of the silicate-based compound in the dispersion liquid may be regulated as appropriate according to the type of the silicate-based compound or the like; however, for example, the content is preferably 0% to 90% by mass, and more preferably 0% to 80% by mass, with respect to the total solid content of the dispersion liquid.

In a case in which the dispersion liquid includes a compound represented by General Formula (1) as the silicate-based compound, it is preferable that the dispersing medium is water. It is because by using water as a dispersing medium, the burden on the health of workers at the time of handling and the burden on the environment are reduced, and also, a hydrolysate of the compound represented by General Formula (1) can be prevented from being condensed in the liquid during storage.

<Method for Producing Dispersion Liquid>

The method for producing the dispersion liquid is not particularly limited, and for example, the dispersion liquid is obtained by mixing the essential components and optional components described above as appropriate.

The content of the total solid content mass with respect to the total mass of the dispersion liquid in the dispersion liquid is preferably 50% by mass or less, and more preferably 40% by mass or less, from the viewpoint of having superior resistance to settling. The lower limit is not particularly limited; however, for example, the lower limit is 0.00001% by mass or more.

<Physical Properties of Dispersion Liquid>

(pH of Dispersion Liquid)

The pH of the dispersion liquid is not particularly limited; however, in a case in which rough hands of the use in an actual use environment and the like are considered, it is preferable to adjust the pH to an appropriate range.

The pH of the dispersion liquid is preferably 2 to 12, and more preferably 3 to 11. Meanwhile, the pH can be measured using a commercially available pH measuring meter (for example, pH meter HM-30R manufactured by DKK-Toa Corporation).

(Viscosity of Dispersion Liquid)

The viscosity of the dispersion liquid is not particularly limited. Of course, in a case in which the viscosity is high, settling of the composite particles can be further suppressed, while the use suitability may be deteriorated. Therefore, it is preferable to adjust the viscosity to an appropriate range.

From such a viewpoint, in the case of considering coatability or spray suitability, the viscosity at 25° C. of the dispersion liquid is preferably 300 cP (centipoises: 1 cp=1 mPa·s) or lower, more preferably 200 cP or lower, and even more preferably 0.1 to 150 cP.

Furthermore, in the case of considering long-term storage for space deodorization, the viscosity at 25° C. of the dispersion liquid is preferably 250 cP or higher, more preferably 300 cP or higher, and even more preferably 400 cP or higher.

The viscosity can be measured using VISCOMETER TUB-10 manufactured by Toki Sangyo Co., Ltd., or SEKONIC VISCOMETER manufactured by Sekonic Corporation.

(Zeta Ppotential)

The zeta potential of the dispersion liquid is not particularly limited; however, as it is considered that superior resistance to settling is obtained in a case in which the composite particles are appropriately dispersed, and a desired particle diameter with suppressed aggregation is obtained, it is preferable to adjust the zeta potential to an appropriate range.

The zeta potential of the dispersion liquid according to the embodiment of the invention is preferably 80 mV to −80 mV, more preferably 70 mV to −70 mV, and even more preferably 60 mV to −60 mV.

The zeta potential can be measured using a known method. A predetermined amount of a dispersion liquid is introduced into a glass measurement cell for exclusive use, the zeta potential can be measured using ELSZ1EAS manufactured by Otsuka Electronics Co., Ltd.

(Surface Tension)

The surface tension of the dispersion liquid is not particularly limited; however, as wettability at the time of applying a dispersion liquid to the coating use is considered, it is preferable to adjust the surface tension to an appropriate range.

The surface tension of the dispersion liquid according to the embodiment of the invention is preferably 300 mN/m or less, more preferably 200 mN/m or less, and even more preferably 100 mN/m or less. Meanwhile, the lower limit is not particularly limited; however, for example, the lower limit is 5 mN/m or more.

The surface tension can be measured using a surface tension meter, DY-300, manufactured by Kyowa Interface Science Co., Ltd.

[Film]

A film according to the embodiment of the invention has composite particles and a binder.

The film can be formed using a dispersion liquid including the above-mentioned film-forming agent.

In the following description, the film according to the embodiment of the invention will be explained using a film (coating film) formed using the dispersion liquid as an example. The film (coating film) can be formed by, for example, applying the dispersion liquid on a base material and drying the dispersion liquid.

<Base Material>

The base material on which the dispersion liquid is applied is not particularly limited, and a glass base material, a resin base material, a metal base material, a ceramic base material, a fabric, and the like are used as appropriate.

Examples of the resin that constitutes the resin base material include polypropylene, polystyrene, polyurethane, an acrylic resin, polycarbonate, polyamide, a fluororesin, a latex, polyvinyl chloride, a polyolefin, a melamine resin, an ABS (acrylonitrile-butadiene-styrene) resin, and a polyester (for example, polyethylene terephthalate (PET)).

The shape of the base material is not particularly limited, and examples thereof include a plate shape, a film shape, and a sheet shape. Furthermore, the base material surface may be a flat surface, a concave surface, or a convex surface. On the surface of the base material, an easily adhesive layer known in the related art may be formed.

<Method for Forming Coating Film>

The method for applying the dispersion liquid is not particularly limited, and examples thereof include a spraying method, a brush coating method, an immersion method, an electrostatic painting method, a bar coating method, a roll coating method, a flow coating method, a die coating method, a nonwoven fabric coating method, an inkjet method, a casting method, a spin coating method, and a Langmuir-Blodgett (LB) method.

Drying after application may be drying at room temperature, or may be heating at 40° C. to 120° C. The drying time is, for example, about 1 to 30 minutes.

<Film Thickness>

The film thickness of the film is not particularly limited; however, the film thickness is preferably 10,000 nm or less, more preferably 1 to 5,000 nm, and even more preferably 3 to 1,000 nm.

[Use Applications]<Deodorizing Material>

A deodorizing material according to the embodiment of the invention includes the above-described composite particles, the above-described dispersion liquid, or the above-described film.

The inorganic particles included in the composite particles, the dispersion liquid, or the film has an average particle diameter of less than 100 nm, and for example, the inorganic particles have a deodorizing effect by physical adsorption or chemical reaction of components such as hydrogen sulfide. Therefore, the composite particles, the dispersion liquid, or the film can be used as deodorizing material.

In the following description, aspects of using the composite particles, the dispersion liquid, or the film as a deodorizing material will be respectively explained.

(Aspect of Using Dispersion Liquid as Deodorizing Material)

The dispersion liquid described above can be used as a deodorizing material. That is, a deodorization treatment can be carried out using the dispersion liquid as a deodorizing material. In the present specification, a deodorization treatment means deodorizing a space or an article using the dispersion liquid described above.

Examples of the deodorization treatment include, specifically, a treatment of deodorizing an article by forming a film containing composite particles on the article; a treatment of deodorizing a space by spraying a dispersion liquid containing composite particles in the space; and a treatment of deodorizing a space by leaving a dispersion liquid containing composite particles to stand in a free state.

Regarding a treatment of deodorizing an article by forming a film containing composite particles on an article, for example, wipe coating of impregnating a base fabric (for example, a nonwoven fabric) with the dispersion liquid, and then wiping and stretching the composite particles on the surface of the article using the nonwoven fabric, may be mentioned as an aspect. Furthermore, an aspect in which the dispersion liquid is accommodated in a spray container capable of spraying a liquid, such as a spray can, and the dispersion liquid is applied by spraying the dispersion liquid on the surface of the article (hand spray application), is also acceptable. A film containing the composite particles is formed on an article by wipe application and hand spray application.

The article is not particularly limited; however, for example, the article is an article in need of deodorization, and specific examples thereof include facilities such as an electronic instrument and a medical instrument, and construction materials such as a bed, a wall, and a handrail. The facilities may be facilities that have already been installed, or may be already in operation.

Regarding a treatment of deodorizing a space by spraying a dispersion liquid containing composite particles in the space, specifically, for example, an aspect of accommodating the dispersion liquid in a spray container capable of spraying a liquid, such as a spray can, and spraying the dispersion liquid in the space, may be mentioned.

Regarding a treatment of deodorizing a space by leaving a dispersion liquid containing composite particles to stand in a free state, specifically, for example, an aspect of deodorizing a space by accommodating the dispersion liquid in a container having an opening, and then leaving the container accommodating the dispersion liquid to stand in a space in need of deodorization, may be mentioned.

Meanwhile, the dispersion liquid can also be used for antibacterial, antiviral, and antifungal use applications in addition to the use for deodorization.

(Aspect of Using Film as Deodorizing Material)

The film can be used as a deodorizing material. The film as used herein means a film having composite particles and a binder.

In the case of using a film as a deodorizing material, the film itself can be used as a deodorizing sheet. Regarding a method of disposing a deodorizing sheet in various apparatuses, for example, a film may be formed by directly applying the above-described dispersion liquid on the surface of the above-mentioned article, or a film may be separately formed and adhered on the surface of the above-mentioned article through a pressure sensitive adhesive layer or the like.

(Aspect of Using Composite Particles as Deodorizing Material)

The composite particles can be used as a deodorizing material. In the case of using the composite particles as a deodorizing material, the composite particles themselves can be used as a deodorizing material.

[Wet Wiper]

A wet wiper according to the embodiment of the invention has a base fabric and the above-described dispersion liquid impregnated into the base fabric. The wet wiper according to the embodiment of the invention can be used per se as a wet wiper having deodorizing properties. Furthermore, the dispersion liquid can be applied on the surface of a base material using the wet wiper according to the embodiment of the invention.

In the following description, the wet wiper according to the embodiment of the invention will be explained. The dispersion liquid is as described above.

The base fabric used for the wet wiper is not particularly limited, and the base fabric may be a fabric formed from natural fibers or a fabric formed from chemical fibers.

Examples of the natural fibers include pulp, cotton, hemp, flax, wool, camel, cashmere, mohair, and silk.

Examples of the material for the chemical fibers include rayon, polynosic, acetate, triacetate, nylon, polyester, polyacrylonitrile, polyvinyl alcohol, polyvinyl chloride, polyvinylidene chloride, polyethylene, polypropylene, polyurethane, polyalkylene para-oxybenzoate, and polychlal.

Above all, among these base fabrics, from the viewpoint of being easily impregnated with a dispersion liquid containing composite particles, a hydrophilic base fabric is preferred. A hydrophilic base fabric is a base fabric including fibers containing hydrophilic groups such as a hydroxyl group, an amino group, a carboxyl group, an amide group, and a sulfonyl group. Specific examples of the hydrophilic base fabric include plant fibers, cotton, pulp, animal fibers, rayon, nylon, polyester, polyacrylonitrile, and polyvinyl alcohol.

Examples of the base fabric for the wet wiper include a nonwoven fabric, a fabric, a towel, gauze, and decreased cotton, and among them, a nonwoven fabric is preferred.

The basis weight (mass per unit area) of the base fabric is preferably 100 g/m² or less. The amount of impregnation at the time of impregnating the base fabric with the dispersion liquid is preferably an amount one or more times the mass of the base fabric.

The content of the composite particles in the wet wiper is not particularly limited; however, from the viewpoint of having superior deodorizing properties, generally, the content is preferably 100 to 5,000 parts by mass, more preferably 500 to 5,000 parts by mass, and even more preferably 1,000 to 5,000 parts by mass, with respect to 100 parts by mass of the base fabric.

[Spray]

A spray according to the embodiment of the invention comprises a spray container and the above-described dispersion liquid stored in the spray container. Specifically, the spray can be formed by charging the dispersion liquid and a propellant into a predetermined container. The propellant to be used is not particularly limited; however, examples thereof include liquefied petroleum gas.

### EXAMPLES

Hereinafter, the invention will be described in more detail based on Examples. The materials, amount of use, proportions, content of treatment, treatment procedure, and the like shown in the following Examples can be modified as appropriate as long as the purport of the invention is maintained. Therefore, the scope of the invention is not intended to be interpreted limitedly by the Examples described below.

### Example 1

<Production of Dispersion Liquid 1>

Copper oxide particles (“copper(II) oxide COPPER OXIDE” manufactured by Kanto Chemical Co., Inc.) were dried at low temperature under reduced pressure under the conditions of 4° C. and 40 hours, and thereby, moisture was removed. Next, the copper oxide particles after drying were dispersed by diluting the particles 10 times with water, and then the copper oxide particles were wet-pulverized using a beads mill. The dispersion liquid thus obtained was dried under reduced pressure under the conditions of 50° C. and 5 hours, and thus a CuO powder having an average particle diameter of 30 nm was produced.

For copper(II) oxide particles used in other dispersion liquids shown below, the particle diameter control was carried out by a method similar to that for the copper(II) oxide used in the dispersion liquid 1, except that the milling time and the type of filter were changed. Furthermore, also for copper(I) oxide used in dispersion liquid 7, zinc oxide used in dispersion liquid 11, copper used in dispersion liquid 16, and silver oxide used in dispersion liquid 17 and dispersion liquid 21, particle diameter control was carried out according to the method described above.

In a container, while 150 g of an aqueous dispersion liquid (solid content concentration: 0.1% by mass) of polymer particles (“EPOSTAR 100W” manufactured by Nippon Shokubai Co., Ltd.) was stirred, 0.1 g of a silicate-based compound (“MKC (registered trademark) SILICATE MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 30 nm) of copper oxide (“copper(II) oxide COPPER OXIDE” manufactured by Kanto Chemical Co., Inc.) that had been subjected to particle diameter control was added, and the mixture was stirred for another 20 minutes. Thus, dispersion liquid 1 was obtained. The dispersion liquid 1 thus obtained was designated as Example 1.

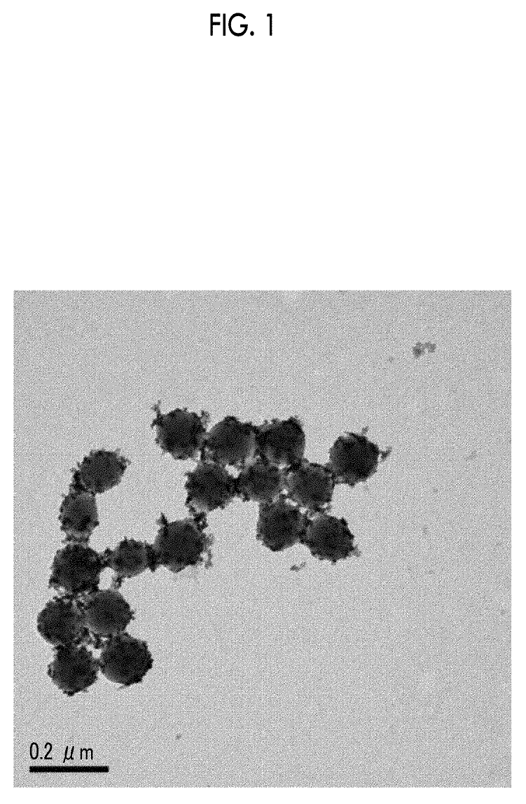

The dispersion liquid thus obtained was centrifuged, and thereby the composite particles were settled down. The composite particles were separated by filtration and were dried naturally under reduced pressure, and thereby composite particles 1 were obtained. An optical microscopic photograph of the composite particles 1 is shown in FIG. 1. As is obvious from FIG. 1, the composite particles 1 have a structure in which copper oxide particles are supported on the surface of polymer particles. It is speculated that a coating film of a silane compound obtained by condensing a silicate-based compound is formed in at least one region on the surface of polymer particles.

In the present Examples, regarding the average particle diameters of the inorganic particles and the polymer particles, the average particle diameters obtained by measurement based on dynamic light scattering using a dispersion liquid of the inorganic particles only and a dispersion liquid of the polymer particles only were used instead. The specific method is as described above.

<Evaluation>

(Deodorization Test)

For the dispersion liquid 1 obtained as described above, an evaluation of the deodorizing properties of the dispersion liquid was carried out by determining the H₂S removal ratio (%) by the following method.

The H₂S removal ratio was obtained by leaving a filter paper coated with the dispersion liquid 1 to stand in a Tedlar bag filled with an odorous gas, and calculating the removal ratio by the following expression from the measured values of the H₂S concentration before and after the standing. Specific measurement conditions and measurement method will be described below.

“H₂S removal ratio={(Initial H₂S concentration in ppm)−(concentration of H₂S remaining after standing in ppm)}/(initial H₂S concentration in ppm)×100”

<<Specific Measurement Conditions and Measurement Method for H₂S Removal Ratio>>

Coating amount of inorganic particles 1 in the dispersion liquid: 0.1 mg in 100 cm²

Testing method, standards: JTETC method and detector tube method

Odorous gas species: Hydrogen sulfide, 20 ppm

Diluent gas conditions: Mixing with dry N₂ gas, humidified for 24 hours or longer at 20° C. and a humidity of 65% (according to the provisions of the JTETC method)

Time of exposure to odorous gas: 2 hours

Capacity of the Tedlar bag filled with odorous gas: 3 L

For the filter paper used in the test, a commercially available cellulosic filter paper having a basis weight of 450 g/m² and a thickness of 1.5 mm was used.

(Dispersibility (Settling Properties))

Regarding the evaluation of dispersibility, the dispersion liquid 1 was stirred, and then while the dispersion liquid was left to stand for one week at room temperature, the presence of settling material was checked. The evaluation was performed according to the following criteria. For practical use, a grade of “B” or higher is preferred.

“A”: No settling material seen even after one week.

“B”: No settling material seen for a time period of more than three days and within one week

“C”: Settling material found within three days.

### Examples 2 to 17 and Comparative Examples 1 to 4

Dispersion liquids 2 to 17 and dispersion liquids 18 to 21 were prepared as follows, and the dispersion liquids were designated as Examples 2 to 17 and Comparative Examples 1 to 4, respectively. For all of the dispersion liquids 2 to 17, it was confirmed that similarly to dispersion liquid 1, composite particles having a structure in which inorganic particles were supported on the surface of polymer particles were formed. Furthermore, it is speculated that a coating film of a silane compound obtained by condensing a silicate-based compound is formed in at least one region on the surface of the polymer particle.

Various evaluations were performed by methods similar to the case of dispersion liquid 1, using the dispersion liquids 2 to 21 thus obtained. The results are presented in Table 1.

<Production of Dispersion Liquid 2>

In a container, while 150 g of an aqueous dispersion liquid (solid content concentration: 0.1% by mass) of polymer particles (“MP-2800” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 50 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 2 was obtained.

<Production of Dispersion Liquid 3>

In a container, while 150 g of an ethanol dispersion liquid (solid content concentration: 0.1% by mass) of polymer particles (“MP-2800” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an ethanol dispersion liquid (solid content concentration: 0.01% by mass, average particle diameter: 50 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 3 was obtained.

<Production of Dispersion Liquid 4>

In a container, while 150 g of an aqueous dispersion liquid (solid content concentration: 0.1% by mass, average particle diameter: 103 nm) of polymer particles having the particle diameter controlled (“MP-2800” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 50 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 4 was obtained.

<Production of Dispersion Liquid 5>

In a container, while 150 g of an aqueous dispersion liquid (solid content concentration: 0.1% by mass) of polymer particles (“EPOSTAR 100W” manufactured by Nippon Shokubai Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 40 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 5 was obtained.

<Production of Dispersion Liquid 6>

In a container, while 150 g of an aqueous dispersion liquid (solid content concentration: 0.25% by mass) of polymer particles (“MP-1000” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.02% by mass, average particle diameter: 50 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 6 was obtained.

<Production of Dispersion Liquid 7>

In a container, while 150 g of an aqueous dispersion liquid (solid content concentration: 0.1% by mass) of polymer particles (“MX-80H3wT” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 97 nm) of copper oxide having the particle diameter controlled (“copper(I) oxide nanospheres, dispersion” manufactured by Sigma-Aldrich Corporation.) was added, and the mixture was stirred for another 20 minutes. Thus, dispersion liquid 7 was obtained.

<Production of Dispersion Liquid 8>

In a container, while 150 g of an aqueous dispersion liquid (solid content concentration: 0.1% by mass) of polymer particles (“AROBASE SE-1013N” manufactured by Unitika, Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 97 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 8 was obtained.

<Production of Dispersion Liquid 9>

In a container, while 100 g of an aqueous dispersion liquid (solid content concentration: 0.1% by mass) of polymer particles (“EPOSTAR 050W” manufactured by Nippon Shokubai Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.0025% by mass, average particle diameter: 30 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 9 was obtained.

<Production of Dispersion Liquid 10>

In a container, while 150 g of an aqueous dispersion liquid (solid content concentration: 0.1% by mass) of polymer particles (“SX8743(C)-03” manufactured by JSR Corporation) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 60 nm) of copper oxide having the particle diameter controlled (“copper(II) oxide COPPER OXIDE” manufactured by Kanto Chemical Co., Inc.) was added, and the mixture was stirred for another 20 minutes. Thus, dispersion liquid 10 was obtained.

<Production of Dispersion Liquid 11>

In a container, while 150 g of an aqueous dispersion liquid (solid content concentration: 0.1% by mass) of polymer particles (“EPOSTAR 100W” manufactured by Nippon Shokubai Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 60 nm) of zinc oxide having the particle diameter controlled (“NANOPURE ZINC OXIDE NANOPOWDER” manufactured by Japan Ion Corporation) was added, and the mixture was stirred for another 20 minutes. Thus, dispersion liquid 11 was obtained.

<Production of Dispersion Liquid 12>

In a container, while 100 g of an aqueous dispersion liquid (solid content concentration: 1.0% by mass) of polymer particles (“EPOSTAR 050W” manufactured by Nippon Shokubai Co., Ltd.) was stirred, 1.0 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.025% by mass, average particle diameter: 30 nm) of copper oxide having the particle diameter controlled (“copper(II) oxide COPPER OXIDE” manufactured by Kanto Chemical Co., Ltd.) was added, and the mixture was stirred for another 20 minutes. Thus, dispersion liquid 12 was obtained.

<Production of Dispersion Liquid 13>

In a container, while 100 g of an aqueous dispersion liquid (solid content concentration: 0.1% by mass) of polymer particles (“BONRON PS-002” manufactured by Mitsui Chemicals, Inc.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 30 nm) of copper oxide having the particle diameter controlled (“copper(II) oxide COPPER OXIDE” manufactured by Kanto Chemical Co., Ltd.) was added, and the mixture was stirred for another 20 minutes. Thus, dispersion liquid 13 was obtained.

<Production of Dispersion Liquid 14>

In a container, while 150 g of an aqueous dispersion liquid (solid content concentration: 0.1% by mass) of polymer particles (“MX-80H3wT” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.02% by mass, average particle diameter: 50 nm) of copper oxide having the particle diameter controlled (“copper(II) oxide COPPER OXIDE” manufactured by Kanto Chemical Co., Ltd.) was added, and the mixture was stirred for another 20 minutes. Thus, dispersion liquid 14 was obtained.

<Production of Dispersion Liquid 15>

In a container, while 150 g of an aqueous dispersion liquid (solid content concentration: 0.1% by mass) of polymer particles having the particle diameter controlled (“MX-150” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.02% by mass, average particle diameter: 50 nm) of copper oxide having the particle diameter controlled (“copper(II) oxide COPPER OXIDE” manufactured by Kanto Chemical Co., Ltd.) was added, and the mixture was stirred for another 20 minutes. Thus, dispersion liquid 15 was obtained.

<Production of Dispersion Liquid 16>

In a container, while 150 g of an aqueous dispersion liquid (solid content concentration: 0.1% by mass) of polymer particles (“EPOSTAR 100W” manufactured by Nippon Shokubai Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 60 nm) of copper having the particle diameter controlled (“copper, powder” manufactured by Wako Pure Chemical Industries, Ltd.) was added, and the mixture was stirred for another 20 minutes. Thus, dispersion liquid 16 was obtained.

<Production of Dispersion Liquid 17>

In a container, while 150 g of an aqueous dispersion liquid (solid content concentration: 0.1% by mass) of polymer particles (“EPOSTAR 100W” manufactured by Nippon Shokubai Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 60 nm) of silver oxide having the particle diameter controlled (“silver oxide” manufactured by Wako Pure Chemical Industries, Ltd.) was added, and the mixture was stirred for another 20 minutes. Thus, dispersion liquid 17 was obtained.

<Production of Dispersion Liquid 18>

In a container, while 150 g of an aqueous dispersion liquid (solid content concentration: 0.1% by mass, average particle diameter: 50 nm) of polymer particles having the particle diameter controlled (“MP-2800” manufactured by Soken Chemical & Engineering Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 300 nm) of copper oxide (“copper(II) oxide COPPER OXIDE” manufactured by Kanto Chemical Co., Ltd.) was added, and the mixture was stirred for another 20 minutes. Thus, dispersion liquid 18 was obtained.

<Production of Dispersion Liquid 19>

In a container, while 150 g of an aqueous dispersion liquid (solid content concentration: 0.1% by mass) of polymer particles (“EPOSTAR 050W” manufactured by Nippon Shokubai Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 110 nm) of copper oxide having the particle diameter controlled (“copper(II) oxide COPPER OXIDE” manufactured by Kanto Chemical Co., Ltd.) was added, and the mixture was stirred for another 20 minutes. Thus, dispersion liquid 19 was obtained.

<Production of Dispersion Liquid 20>

In a container, an aqueous dispersion liquid (solid content concentration: 0.01% by mass, average particle diameter: 60 nm) of copper oxide having the particle diameter controlled (“copper(II) oxide COPPER OXIDE” manufactured by Kanto Chemical Co., Ltd.) was stirred for 20 minutes. Thus, dispersion liquid 20 was obtained.

<Production of Dispersion Liquid 21>

In a container, while 150 g of an aqueous dispersion liquid (solid content concentration: 0.1% by mass) of polymer particles (“EPOSTAR 100W” manufactured by Nippon Shokubai Co., Ltd.) was stirred, 0.1 g of a silicate-based compound (“MKC (registered trademark) Silicate MS51” manufactured by Mitsubishi Chemical Corporation) was added thereto, and the mixture was stirred for 20 minutes. Next, to this stirred product, 50 g of an aqueous dispersion liquid (solid content concentration: 0.01% by mass, average particle diameter: 300 nm) of silver oxide having the particle diameter controlled (“silver oxide” manufactured by Wako Pure Chemical Industries, Ltd.) was added, and the mixture was stirred for another 20 minutes. Thus, dispersion liquid 21 was obtained.

TABLE 1

| | Dispersion liquid | | | | | | | |
|---|---|---|---|---|---|---|---|---|
| | | Average | Average | | | | Evaluation | |
| | | particle | particle | | | | Deodorizing | |
| | | diameter of | diameter of | | | | properties | |
| | Type of | polymer | inorganic | | Type of | | H₂S removal | Dispersibility |
| | dispersion | particles | particles | Type of polymer | inorganic | | ratio | (resistance to |
| | liquid | (nm) | (nm) | particles | particles | Solvent | (%) | settling) |
| Example 1 | Dispersion | 150 | 30 | (Meth)acrylic resin | CuO | Water | 85 | A |
| | liquid 1 | | | | | | | |
| Example 2 | Dispersion | 200 | 50 | (Meth)acrylic resin | CuO | Water | 70 | A |
| | liquid 2 | | | | | | | |
| Example 3 | Dispersion | 200 | 50 | (Meth)acrylic resin | CuO | Alcohol | 70 | A |
| | liquid 3 | | | | | | | |
| Example 4 | Dispersion | 103 | 50 | (Meth)acrylic resin | CuO | Water | 68 | A |
| | liquid 4 | | | | | | | |
| Example 5 | Dispersion | 150 | 40 | (Meth)acrylic resin | CuO | Water | 75 | A |
| | liquid 5 | | | | | | | |
| Example 6 | Dispersion | 400 | 50 | (Meth)acrylic resin | CuO | Water | 71 | A |
| | liquid 6 | | | | | | | |
| Example 7 | Dispersion | 800 | 97 | (Meth)acrylic resin | Cu₂O | Water | 56 | B |
| | liquid 7 | | | | | | | |
| Example 8 | Dispersion | 94 | 97 | Polyolefin resin | CuO | Water | 53 | B |
| | liquid 8 | | | | | | | |
| Example 9 | Dispersion | 67 | 30 | (Meth)acrylic resin | CuO | Water | 85 | B |
| | liquid 9 | | | | | | | |
| Example 10 | Dispersion | 300 | 60 | Polystyrene resin | CuO | Water | 66 | A |
| | liquid 10 | | | | | | | |
| Example 11 | Dispersion | 150 | 60 | (Meth)acrylic resin | ZnO | Water | 65 | A |
| | liquid 11 | | | | | | | |
| Example 12 | Dispersion | 67 | 30 | (Meth)acrylic resin | CuO | Water | 85 | B |
| | liquid 12 | | | | | | | |
| Example 13 | Dispersion | 250 | 30 | Polystyrene- | CuO | Water | 85 | A |
| | liquid 13 | | | (meth)acrylic | | | | |
| | | | | copolymer resin | | | | |
| Example 14 | Dispersion | 800 | 50 | (Meth)acrylic resin | CuO | Water | 71 | A |
| | liquid 14 | | | | | | | |
| Example 15 | Dispersion | 1000 | 50 | (Meth)acrylic resin | CuO | Water | 69 | B |
| | liquid 15 | | | | | | | |
| Example 16 | Dispersion | 150 | 60 | (Meth)acrylic resin | Cu | Water | 60 | A |
| | liquid 16 | | | | | | | |
| Example 17 | Dispersion | 150 | 60 | (Meth)acrylic resin | Ag₂O | Water | 35 | A |
| | liquid 17 | | | | | | | |
| Comparative | Dispersion | 50 | 300 | (Meth)acrylic resin | CuO | Water | 10 | C |
| Example 1 | liquid 18 | | | | | | | |
| Comparative | Dispersion | 67 | 110 | (Meth)acrylic resin | CuO | Water | 32 | C |
| Example 2 | liquid 19 | | | | | | | |
| Comparative | Dispersion | None | 60 immediately | — | CuO | Water | 21 | C |
| Example 3 | liquid 20 | | after dispersing | | | | | |
| Comparative | Dispersion | 150 | 300 | (Meth)acrylic resin | Ag₂O | Water | 7 | C |
| Example 4 | liquid 21 | | | | | | | |

From the results shown in Table 1, it was confirmed that the dispersion liquids of the Examples have excellent deodorizing properties as well as excellent resistance to settling.

Furthermore, from a comparison of Examples 1, 5, and 11, it was confirmed that in a case in which the average particle diameter of the inorganic particles is 50 nm or less, superior deodorizing properties are obtained.

From a comparison of Examples 1, 9, and 12, it was confirmed that in a case in which the average particle diameter of the polymer particles is 100 nm or more, superior resistance to settling is obtained. From a comparison of Examples 14 and 15, it was confirmed that in a case in which the average particle diameter of the polymer particles is 800 nm or less, superior resistance to settling is obtained.

From a comparison of Examples 9 and 12, it was confirmed that even in a case in which the concentration of the composite particles with respect to the total mass of the dispersion liquid varies, there is no variation in the deodorizing properties and the resistance to settling.

Furthermore, in Examples 7 and 8, it is speculated that since the inorganic microparticles have a large size with an average particle diameter of more than 90 nm, it is difficult for the inorganic particles to be supported on the polymer particles, and the results for resistance to settling were rated as “B”.

Meanwhile, the dispersion liquids of Comparative Examples did not satisfy desired requirements for any of deodorizing properties or resistance to settling.

What is claimed is:

1. A composite particle comprising:
a polymer particle; and
at least one kind of inorganic particle selected from the group consisting of metal particles and metal oxide particles, the inorganic particle being supported on the surface of the polymer particle,
wherein the inorganic particles have an average particle diameter of less than 100 nm.

2. The composite particle according to claim 1, further comprising a coating film formed from a silane compound on at least a portion of the surface of the polymer particle.

3. The composite particle according to claim 1, wherein the inorganic particle includes at least one selected from the group consisting of Cu, Ag, Zn, Ti, Ni, W, Sn, Fe, Sr, Bi, and Mn.

4. The composite particle according to claim 1, wherein the polymer particles have an average particle diameter of more than 50 nm.

5. The composite particle according to claim 1, wherein the polymer particles have an average particle diameter of 100 to 800 nm.

6. The composite particle according to claim 1, wherein the polymer particle includes, as a resin material that constitutes the polymer particle, at least one selected from the group consisting of an acrylic resin, a methacrylic resin, a polystyrene resin, a polyolefin resin, and a copolymer formed from a polystyrene resin and an acrylic resin or a methacrylic resin.

7. A dispersion liquid comprising:
the composite particle according to claim 1; and
a solvent.

8. The dispersion liquid according to claim 7, further comprising a thermoplastic resin or a silicate-based compound.

9. A film comprising:
the composite particle according to claim 1; and
a binder.

10. A deodorizing material comprising the composite particle according to claim 1.

11. A wet wiper comprising:
a base fabric; and
the dispersion liquid according to claim 7 impregnated into the base fabric.

12. A spray comprising:
a spray container; and
the dispersion liquid according to claim 7 stored in the spray container.

13. A deodorizing material comprising the dispersion liquid according to claim 7.

14. A deodorizing material comprising the film according to claim 9.

15. The composite particle according to claim 2, wherein the inorganic particle includes at least one selected from the group consisting of Cu, Ag, Zn, Ti, Ni, W, Sn, Fe, Sr, Bi, and Mn.

16. The composite particle according to claim 2, wherein the polymer particles have an average particle diameter of more than 50 nm.

17. The composite particle according to claim 2, wherein the polymer particles have an average particle diameter of 100 to 800 nm.

18. The composite particle according to claim 3, wherein the polymer particles have an average particle diameter of 100 to 800 nm.

19. The composite particle according to claim 2, wherein the polymer particle includes, as a resin material that constitutes the polymer particle, at least one selected from the group consisting of an acrylic resin, a methacrylic resin, a polystyrene resin, a polyolefin resin, and a copolymer formed from a polystyrene resin and an acrylic resin or a methacrylic resin.

20. The composite particle according to claim 5, wherein the polymer particle includes, as a resin material that constitutes the polymer particle, at least one selected from the group consisting of an acrylic resin, a methacrylic resin, a polystyrene resin, a polyolefin resin, and a copolymer formed from a polystyrene resin and an acrylic resin or a methacrylic resin.
